SEQUENCE LISTING (11 sequences)

SEQ ID NO: 1 - protein, 9 aa, Human immunodeficiency virus type 1
SLYNTVATL

SEQ ID NO: 2 - protein, 9 aa, Human immunodeficiency virus type 1
EIYKRWIIL

SEQ ID NO: 3 - protein, 10 aa, Human immunodeficiency virus type 1
KAALDLSHFL

SEQ ID NO: 4 - protein, 11 aa, Human herpesvirus 4
FIAGNSAYEYV

SEQ ID NO: 5 - protein, 12 aa, Human herpesvirus 4
SDEEEAIVAYTL

SEQ ID NO: 6 - protein, 9 aa, Human herpesvirus 5
RPPIFIRRL

SEQ ID NO: 7 - protein, 9 aa, Human herpesvirus 5
EGGVGWRHV

SEQ ID NO: 8 - protein, 9 aa, Human herpesvirus 5
RLRAEAGVK

SEQ ID NO: 9 - protein, 9 aa, Human herpesvirus 5
IVTDFSVIK

SEQ ID NO: 10 - protein, 11 aa, Human herpesvirus 5
QPRAPIRPIPT

SEQ ID NO: 11 - protein, 10 aa, Human herpesvirus 5
EENLLDFVRF

(19) United States
(12) Patent Application Publication
(10) Pub. No.: US 2008/0213818 A1
(43) Pub. Date: Sep. 4, 2008

(54) CELLULAR IMMUNITY TEST WITH PEPTIDES ATTACHED TO A SOLID SUPPORT

(75) Inventors: Hanne GAHERY-SEGARD, Paris (FR); Jean-Gerard GUILLET, Paris (FR)

Correspondence Address: OBLON, SPIVAK, MCCLELLAND MAIER & NEUSTADT, P.C., 1940 DUKE STREET, ALEXANDRIA, VA, 22314, US

(73) Assignee: INST. NAT. DE LA SANTE ET DE LA RECHERCHE MEDICALE, Paris (FR)

(21) Appl. No.: 11/968,849

(22) Filed: Jan. 3, 2008

Related U.S. Application Data
(63) Continuation of application No. 10/487,890, filed on Feb. 23, 2005, now abandoned.

(30) Foreign Application Priority Data
Aug. 27, 2001 (FR) 01/11136

(51) Int. Cl. C12Q 1/02

(52) U.S. Cl. 435/29

(57) ABSTRACT

The invention concerns a method for detecting cellular immunity (with respect to an antigen), using a solid support whereon is fixed an assortment of peptides constituting T cell epitopes of the antigen to be tested, kits for implementing said method.

### FIELD OF THE INVENTION

The invention relates to novel means for detecting cellular immunity.

### DISCUSSION OF RELATED ART

Cell-mediated immunity plays a major role in the immune response with respect to microorganisms such as viruses, and also certain bacteria or certain protozoa which are capable of developing inside the host's cells, thus escaping antibodies. It is also involved in phenomena of transplant rejection and tumor destruction.

While, in the case of humoral immunity, the molecular effectors are antibodies secreted by B lymphocytes, the molecular effectors of cellular immunity are TcR receptors anchored in the cytoplasmic membrane of T lymphocytes.

The recognition of a T epitope by the TcR involves a mechanism which is much more complex than that which is involved in the recognition of a B epitope by an antibody. Specifically, the TcR will only recognize the peptide constituting its epitope if said epitope is associated with a class I or class II molecule of the MHC (major histocompatibility complex) at the surface of a presenting cell. The TcRs of CD4 and CD8 T lymphocytes thus recognize the epitopes presented respectively by the class II molecules and by the class I molecules of the MHC.

A “T epitope” of an antigen is defined as any peptide fragment of said antigen capable of being recognized, in the context of an appropriate MHC, by the TcR receptor of a T lymphocyte, and of inducing the activation of said lymphocyte.

The size of a T epitope varies depending on the class of the MHC molecule presenting the peptide; CD8⁺ T epitopes presented by class I MHC molecules are 8 to 10 amino acids in size, while CD4⁺ T epitopes presented by class II MHC molecules are 13 to 25 amino acids in size.

For the same antigen and for the same MHC class, the sequence of the epitopes varies according to the allele of the MHC concerned.

For a large number of antigens, a great variety of T epitopes restricted to various class II MHC or class I MHC alleles has been identified and the sequences of these epitopes are available in the literature.

The TcR/MHC-associated T epitope interaction induces, in the presence of various costimulation signals which result from the interaction of molecules at the surface of T cells and molecules at the surface of presenting cells, lymphocyte stimulation resulting in a cell multiplication which produces effector T lymphocytes.

Effector lymphocytes can be grouped together in various populations according to their effector functions, which define various types of immune response. These populations can be distinguished in particular by their ability to secrete various molecules (cytokines, lytic factors) or specific molecule combinations. For example, among effector lymphocytes which can differentiate from CD4⁺ or CD8⁺ cells, mention will be made of Th1 or T1 lymphocytes, which are characterized by the secretion of cytokines, in particular interleukin 2 (IL-2) and gamma-interferon (γ-IFN), and Th2 or T2 cells, which secrete cytokines such as IL-4, IL-5, IL-6 and IL-10.

Unlike humoral immunity, which is significantly detectable about 1 month after first contact with an immunogen, cell-mediated immunity appears over a few days after this first contact; it then persists for a long period of time, in the form of memory T lymphocytes, which are capable of proliferating and of rapidly acquiring effector functions during a subsequent presentation of the same immunogen.

Cellular immunity can therefore constitute a good indicator, allowing very early detection of an immune response with respect to a given antigen, and determination of the type of immune response involved.

The detection of antigen-specific effector lymphocytes is used in various research laboratories, for example in the context of the development of vaccines, to search for immunizing epitopes and to evaluate the strength and the type of the immunogenic response.

An approach which is increasingly commonly used to evaluate cellular immunity with respect to an antigen consists in providing peptides constituting known T epitopes of said antigen and in analyzing the T lymphocyte reactivity with respect to these peptides.

This approach can theoretically be used for diagnosing or monitoring pathologies producing a cell-mediated response. However, unlike humoral immunity, which can be readily detected in vitro by demonstrating the formation of an antigen/antibody complex, the detection of cellular immunity involves the use of laborious techniques which are difficult to use in the context of routine tests.

It is in fact necessary:

1) to present the test epitopes to the lymphocytes, under conditions which allow recognition of said epitopes by the TcR and activation of the lymphocytes;

2) to detect the activated effector lymphocytes.

Some recently developed methods make it possible to simplify effector cell detection. By way of example, the ELISPOT technique, initially described by VERSTEEGEN et al. (J. Immunol. Methods, 111, 25-29, 1988), makes it possible to detect and characterize epitope-specific effector T lymphocytes present among peripheral blood cells by detecting the factors secreted by said lymphocytes after restimulation with peptides representing T epitopes.

For example, this technique has been used to identify CD8⁺ effector T lymphocytes specific for epitopes of the influenza virus (LALVANI et al., J. Exp. Med. 186, 859-865, 1997); to analyze the CD8+ T response with respect to peptides representing various HIV1 epitopes used in vaccines (GAHERY-SEGARD et al., J. Virol., 74, 1694-1703, 2000); to detect the presence of a Mycobacterium tuberculosis infection by detecting effector T lymphocytes specific for epitopes of the ESAT-6 antigen (LALVANI et al., Am. J. Respir. Crit. Care Med., 163, 824-828, 2001).

The assays described in the publications mentioned above were carried out by adding a solution of each of the test peptides to a suspension obtained by separation of peripheral blood mononuclear cells (PBMCs) on a Ficoll gradient; it is in fact known that, under these conditions, these peptides can be loaded directly by the MHC molecules and presented to the lymphocytes.

### SUMMARY OF THE INVENTION

Now, the inventors have now noted that, surprisingly, bringing the T lymphocytes of an individual into contact with peptides constituting epitopes recognized by said T lymphocytes, attached to a solid support, results in activation of effector T lymphocytes specific for said epitopes.

### BRIEF DESCRIPTION OF THE DRAWINGS

FIG. 1 The results of the selection of activated CD8+ T-lymphocytes in Example 2. A: conventional ELISPOT assay; B: assay carried out with the peptides attached to a solid support.

FIG. 2 The results of the characterization of a cellular immunity to CMV in Example 3. A: conventional ELISPOT assay; B: assay carried out with the peptides attached to a solid support.

FIG. 3 The results of the conservation of the peptides attached to a solid support in Example 4 A: conservation at 20° C. for 24 h; B: conservation at 4° C. for 7 days.

FIG. 4 The results of the detection of cellular immunity to EBV using a mixture of peptides attached to a solid support in Example 5:

[symbol] : − without peptide

[symbol] : 10 micrograms of peptide

### DETAILED DESCRIPTION OF THE INVENTION

A subject of the present invention is the use of a solid support to which at least one peptide constituting a T epitope of an antigen is attached, for detecting and/or characterizing, in vitro, the cellular immunity with respect to said antigen.

A subject of the present invention is in particular a method for detecting and/or characterizing, in vitro, the cellular immune response of an individual with respect to an antigen, by bringing a biological sample comprising CD4⁺ T lymphocytes and/or CD8⁺ T lymphocytes present in the peripheral blood mononuclear cells (PBMCs) of said individual into contact with one or more peptide(s) constituting a T epitope or T epitopes of said antigen capable of being presented by an MHC molecule of said individual, and detecting the effector T lymphocytes activated by this bringing into contact, which method is characterized in that said peptide(s) is(are) attached to a solid support.

The biological sample may consist of a preparation of purified CD4⁺ or CD8⁺ T lymphocytes. It may also consist of a preparation of PBMCs obtained from a blood sample from said individual; advantageously, whole blood may be used.

According to a preferred embodiment of the method in accordance with the invention, at least two peptides constituting two different T epitopes of said antigen, and at least one of which is capable of being presented by an MHC molecule of said individual, are attached to said solid support.

According to an advantageous arrangement of this embodiment, a mixture of said peptides is attached to said solid support.

According to another advantageous arrangement of this embodiment, said solid support consists of a microtitration plate, said peptides being attached in at least two different wells of said plate, and the biological sample is divided up into at least two aliquots, each of which is brought into contact with one of said peptides.

The use of a mixture of peptides makes it possible to rapidly detect the existence of a cell-mediated immune response with respect to the antigen concerned. The separate use of various peptides makes it possible to perform a better analysis of the components of this response.

In all cases, the peptide(s) used can constitute a CD4⁺ T epitope or CD4⁺ T epitopes, or a CD8⁺ T epitope or CD8⁺ T epitopes. It is also possible to simultaneously use a CD4⁺ T epitope or CD4⁺ T epitopes, and a CD8⁺ T epitope or CD8⁺ T epitopes, separately or as a mixture.

The other embodiments of the method in accordance with the invention may be identical to those used in the methods for the in vitro detection of the cellular immune response known in the prior art.

For example, the peptides attached to the solid support and the test biological sample are brought into contact under the same conditions as in the methods of the prior art, where the peptides are present in the liquid phase. Generally, this bringing into contact will be carried out by incubation for 5 to 20 hours at 37° C. in the presence of 5% of CO₂.

Similarly, the detection of the activated effector T lymphocytes can be carried out by conventional methods.

Advantageously, the soluble factors (cytokines or lytic factors) secreted by these lymphocytes will be assayed.

For example, assaying IL-2 or γ-IFN makes it possible to characterize a Th1 or T1 response, whereas assaying IL-4, IL-5, IL-6 or IL-10 makes it possible to characterize a Th2 or T2 response. Other soluble factors which characterize essentially CD8⁺ lymphocytes, such as chemokines (RANTES), or molecules with a lytic function (such as perforin/granzyme), can also be assayed.

Other methods for detecting activated T lymphocytes which can optionally be used in the context of the present invention are, for example, intracellular labeling of cytokines.

Compared to the methods for evaluating cellular immunity known in the prior art, in which the test peptides are added to the cell suspension, the method in accordance with the invention makes it possible to considerably simplify the assay procedure, in particular in the case of analyses involving the use, in parallel, of various peptides, which are conventionally carried out in the wells of a microtitration plate or in a series of tubes.

Specifically, while the techniques of the prior art require the addition to the test cells of a defined peptide or a defined mixture of peptides in each of the wells of the plate or in each tube of the series, it is possible, in the context of the implementation of the present invention, to use plates or tubes prepared in advance, each of the wells of said plates or each of said tubes being coated with the desired peptide or mixture of peptides. This makes it possible to considerably reduce the manipulations required, and therefore to decrease the amount of time required to carry out the assay and to increase its reproducibility by limiting the causes of error.

In addition, the attachment of the peptides to a solid support makes it possible to stabilize said peptides and to protect them from the degradation which occurs rapidly in liquid medium. By way of illustration, in the case of peptides attached by adsorption to the wells of a polystyrene microtitration plate, the peptides conserve their properties of specific activation of T lymphocytes for at least 24 hours at 20° C., at least one week at 4° C., and at least 15 days in a freezer at −80° C., whereas solutions of the same peptides lose their properties in a few hours at 37° C.

Furthermore, the present invention has the advantage of considerably decreasing the concentrations of peptides used, thus allowing a very considerable decrease in costs.

A subject of the present invention is also a kit for carrying out a method in accordance with the invention, comprising an assortment of peptides derived from the same antigen, each of which constitutes a T epitope of said antigen, attached to a solid support.

For the preparation of kits in accordance with the invention, use may be made of solid supports, and of methods for attaching the peptides to said supports, of the same type as those which are conventionally used, in particular for antigen/antibody assaying kits.

For example, use may be made of supports made of plastic materials such as polystyrene, polyethylene or polyvinyl chloride, supports made of nitrocellulose, supports made of silica-based materials, such as glass, etc.

The attachment of the peptides may be carried out by techniques which are also known per se; the choice of the most suitable techniques depends on the support concerned and on the physicochemical properties of the peptides chosen.

It is possible, for example, to synthesize the peptides 5 directly on the support, or to attach presynthesized peptides to said support.

For example, the attachment of the peptides can be carried out by adsorption. The support and the conditions for attachment will of course be chosen so as to avoid desorption of the peptides during the addition of the test biological sample and the incubation with said sample.

Advantageously, the peptides will be attached to the support by covalent bond, directly or via a spacer arm. In this case, the surface of the support can be activated by means of agents such as carbodiimide derivatives, N-hydroxysuccinimide derivatives, sulfosuccinimide derivatives, etc.

Biotinylated peptides attached to a support coated with avidin, can also be used. The support can be in the form of a tube or of a set of tubes, of a microtitration plate, of one or more strips, of beads, etc.

The antigen can in particular be an infectious microorganism (virus, protozoan, bacterium), a tumor antigen, an autoimmune antigen or an allergen. If it is a microorganism, the peptides constituting the assortment can be derived from various antigenic proteins of this organism.

The choice of peptides constituting the assortment depends of course on the antigen concerned, and on the precise use for which the kit is intended. They will be peptides previously identified as constituting CD4⁺ or CD8⁺ T epitopes of the antigen concerned.

Advantageously, this assortment will comprise peptides capable of being presented by different MHC alleles. It may consist of peptides constituting CD4⁺ T epitopes, of peptides constituting CD8⁺ T epitopes, or of a mixture of these two types of epitopes.

The peptides constituting the assortment can be mixed and attached in the form of a mixture to the solid support. When the solid support is a microtitration plate or a set of tubes, various peptides or combinations of peptides can also be attached separately, each peptide or peptide combination being attached to the inside of one-of the wells of the microtitration plate, or to the inside of one of the tubes.

A kit in accordance with the invention may also comprise means for detecting the activated effector T lymphocytes. These means may consist of reagents for assaying the soluble factors produced by said lymphocytes.

The present invention may, for example, be used in human or animal health:

in the context of screening and of monitoring the evolution of infections with various pathogenic agents producing a cellular immune response. By way of examples, mention will be made of: viruses such as HIV (human immunodeficiency virus) 1 and 2, FIV (feline immunodeficiency virus), HTLV-1 (human T lymphocyte virus type I), viruses of the various types of hepatitis, etc.; parasites such as Toxoplasma or Plasmodium; prions, etc.;

in the context of screening and of monitoring the evolution of pathologies of tumor origin. By way of examples, mention will be made of: melanoma, breast cancer, myeloid leukemias, cancers associated with a viral infection, such as cervical cancer associated with human papilloma virus, etc.;

in the context of screening and of monitoring the evolution of autoimmune diseases. By way of example, mention will be made of type I diabetes, multiple sclerosis, etc.;

in the context of screening and of monitoring the evolution of diseases of the allergic type. By way of example, mention will be made of allergens such as grasses, pollens, certain medicinal products.

Whatever the use concerned, the present invention allows, by detecting cell-mediated immunity, a very early diagnosis of the appearance or the evolution of a pathology, before the appearance of clinical signs and before the appearance of the humoral response. This makes it possible to make a more rapid medical decision, to choose a better therapeutic strategy and to have a better chance of success of the treatment for the patients.

The present invention will be understood more clearly from the further description which follows, which refers to nonlimiting examples illustrating its implementation for the characterization of a specific cellular immunity with respect to various viral pathogens.

### EXAMPLE 1

### Attachment of Peptides Constituting T Epitopes to a Solid Support

Various peptides constituting known CD8⁺ epitopes of three different viruses: the HIV-1 virus (human immunodeficiency virus), CMV (cytomegalovirus) and the EBV virus (Epstein Barr virus), were synthesized.

These peptides are as follows:

| HIV-1 virus: | | |
|---|---|---|
| gag 77-85 | (SLYNTVATL, HLA A2) | SEQ ID NO: 1 |
| GAG 260-268 | (EIYKRWILL, HLA A2) | SEQ ID NO: 2 |
| Nef 82-91 | (KAALDLSHFL, HLA A2) | SEQ ID NO: 3 |
| CMV virus: | | |
| GB 619-628 | (FIAGNSAYEYV, HLA A2) | SEQ ID NO: 4 |
| IEI 378-389 | (SDEEEAIVAYTL, HLA B18) | SEQ ID NO: 5 |
| EBV virus: | | |
| EBNA 3A 379- | (RPPIFIRRL, HLA B7) | SEQ ID NO: 6 |
| 387 | | |
| EBNA 3C 163- | (EGGVGWRHV, HLA B844) | SEQ ID NO: 7 |
| 171 | | |

These peptides were attached to the bottom of the wells of a polystyrene plate, using the following protocol.

10 μg of peptide in carbonate buffer were deposited into a 96-well microtitration plate (polystyrene plate, NUNC MAXISORB). The attachment of the peptides is carried out by adsorption for 2 hours at ambient temperature. The unattached peptides are removed by washing twice in PBS.

### EXAMPLE 2

### Characterization of a Cellular Immunity with Respect to HIV-1

PBMCs purified from the blood of a patient (HLA-A2) seropositive for HIV-1 were tested as follows:

### 1) Activation of Lymphocytes:

200 μl (105 cells) of cell suspension in conventional culture medium (RPMI, 10% of BSA) are deposited into each of the wells in which the HIV-1 peptides have been attached, as described in example 1 above.

By way of positive control, an assay was carried out by the conventional ELISPOT method: 200 μl (10⁵ cells) of suspension of the PBMCs from the same patient are deposited into the wells of a NUNC 96-well plate (identical to that to which the peptides are attached) and mixed with 10 μg of gag 77-85, GAG 260-268 or Nef 82-91 peptide in solution in conventional culture medium.

In the 2 assays, PBMCs were also deposited in wells without peptides, as a control.

The plates are incubated overnight at 37° C. in an incubator in the presence of 5% of CO₂.

2) Selection of Activated CD8⁺ T lymphocytes:

The following day, the presence of activated CD8⁺ cells is revealed according to the following protocol:

A 96-well nitrocellulose plate (MILLIPORE) was coated with 100 μl/well of anti-γ-IFN antibodies (MABTECH, 1-D1K), diluted to 1 μg/ml in carbonate buffer, for 2 hours at 37° C.

The plate was then washed in PBS and saturated with RPMI, 10% FCS in a proportion of 100 μl/well, for 2 hours at 37° C.

200 μl of cell suspension activated as described in 1) above are deposited into this plate. The incubation is carried out for 5 hours at 37° C., 5% CO₂.

After 1 wash in 1×PBS and 5 washes in PBS containing 0.05% TWEEN, 100 μl/well of anti-γ-IFN biotin antibody (MABTECH, 7-B6-1-Biotin) are added at 1 μg/ml, and incubation is carried out for 2 hours at ambient temperature.

The plates are washed 5 times in PBS-0.05% TWEEN.

100 μl/well of Extravidin-AKP (SIGMA, No. 2636) diluted to 1/5000 in PBS-0.05% TWEEN, 1% BSA are added. The incubation is carried out for 1 hour at ambient temperature. The plates are again washed 5 times in PBS-0.05% TWEEN.

The detection is carried out using the BIORAD kit (No. 5170-6432).

After ½ hour to 1 hour, the reaction is stopped by washing the plates with water. The plates are air-dried.

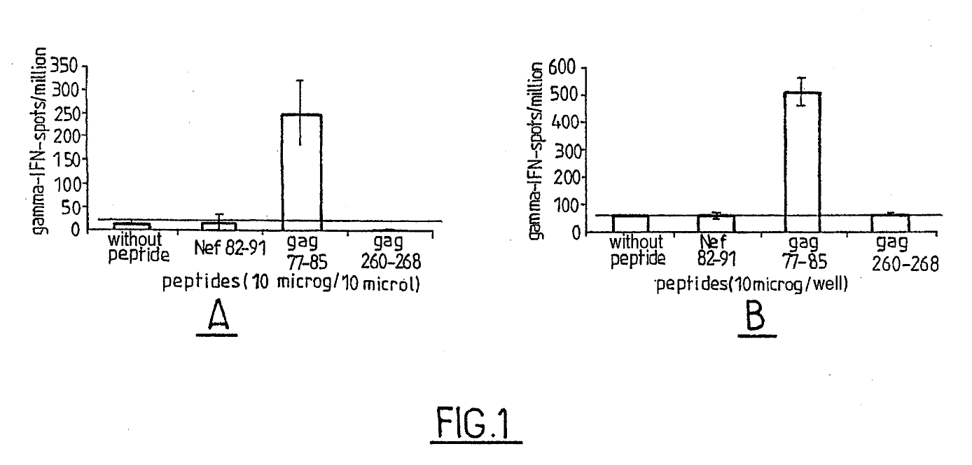

The results are given in FIG. 1.

A: conventional ELISPOT assay;

B: assay carried out with the peptides attached to a solid support.

IN BOTH CASES, ONLY THE GAG 77-85 PEPTIDE INDUCES ACTIVATION OF CD8⁺ EFFECTOR T LYMPHOCYTES SECRETING γ-IFN. THESE RESULTS SHOW THAT THE USE OF PEPTIDES ATTACHED TO A SOLID SUPPORT IS AT LEAST AS EFFECTIVE FOR INDUCING EFFECTOR CELL ACTIVATION AS THAT OF PEPTIDES PRESENTED IN LIQUID PHASE.

### EXAMPLE 3

### Characterization of a Cellular Immunity with Respect to CMV

PBMCs purified from the blood of a patient (HLA-A2, B18) were tested as follows.

2×10⁵ cells in suspension in conventional medium are deposited into each of the wells in which the CMV peptides have been attached, as described in example 1 above.

By way of positive control, an assay was carried out by the conventional ELISPOT method: 2×10⁵ cells of suspension of the PBMCs from the same patient are deposited in the wells of a 96-well plate (NUNC) (identical to that to which the peptides were attached) and mixed with 10 μg of GB 619-628 or IEI 378-389 peptide.

In the 2 assays, PBMCs were also deposited into wells without peptides, as a control.

The incubation and the detection of the plates are carried out as described in example 2 above.

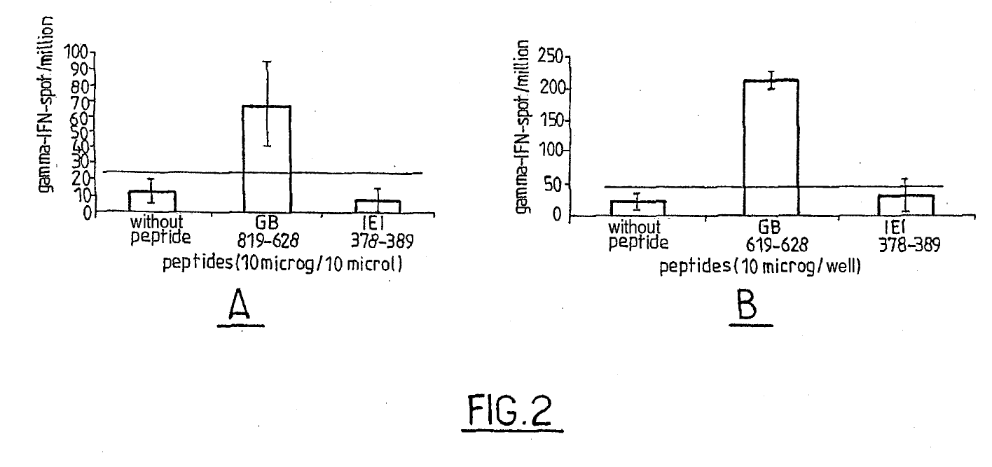

The results are given in FIG. 2.

A: conventional ELISPOT assay;

B: assay carried out with the peptides attached to a solid support.

In both cases, only the GB 619-628 peptide induces activation of CD8⁺ effector T lymphocytes secreting γ-IFN. These results confirm those observed in the case of the HIV-1 peptides.

### EXAMPLE 4

### Conservation of the Peptides Attached to a Solid Support

Plates in the wells of which the EBV peptides have been attached, as described in example 1 above, were conserved:

A) at 20° C. for 24 h;

B) at 4° C. for 7 days;

C) at −80° C. for 15 days.

2×10⁵ PBMCs purified from the blood of a patient (HLA B7/B44), in suspension in conventional culture medium, are deposited into each of the wells of these plates, and tested as described in example 3 above.

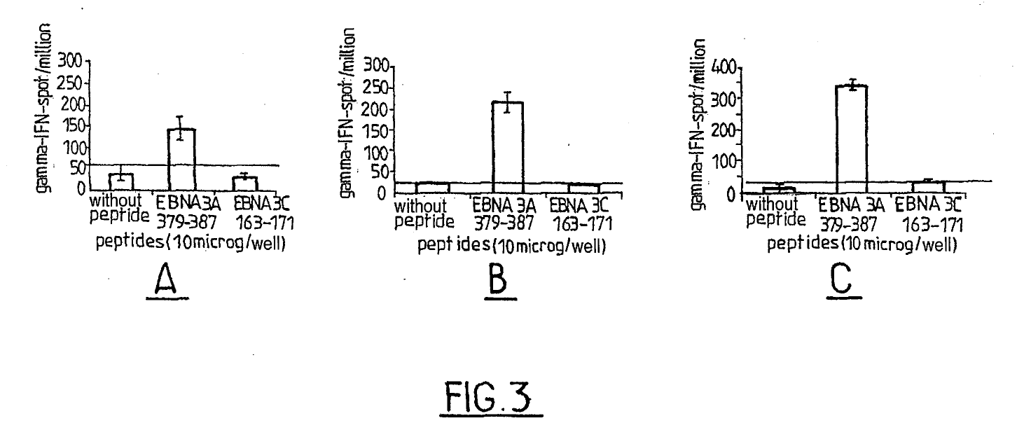

The results are given in FIG. 3.

A: conservation at 20° C. for 24 h;

B: conservation at 4° C. for 7 days;

C: conservation at −80° C. for 15 days.

These results show that the conservation conditions do not modify the effector cell-activating properties of the peptides attached to a solid support.

### EXAMPLE 5

### Detection of a Cellular Immunity with Respect to EBV Using a Mixture of Peptides Attached to a Solid Support

A mixture of the following peptides:

| | | |
|---|---|---|
| EBNA 3C 163-171 | (EGGVGWRHV, B44), | SEQ ID NO: 7 |
| EBNA 3A 603-611 | (RLRAEAGVK, A3), | SEQ ID NO: 8 |
| EBNA 4 416-424 | (IVTDFSVIK, A11), | SEQ ID NO: 9 |
| EBNA 3C 881-891 | (QPRAPIRPIPT, B7) | SEQ ID NO: 10 |
| EBNA 3A 379-387 | (RPPIFIRRL, B7), | SEQ ID NO: 6 |
| and | | |
| EBNA 6 290-299 | (EENLLDFVRF, B44), | SEQ ID NO: 11 |

constituting known CD8⁺ T epitopes of EBV was attached to the inner wall of a polystyrene tube, according to the following protocol:

10 μg of each peptide diluted in carbonate buffer were deposited into a polystyrene tube (15 ml tube, CORNING). The attachment of the peptides is carried out by adsorption for 2 hours at ambient temperature. The unattached peptides are removed by washing twice in PBS.

2 ml of whole blood from a patient (HLA: A3-A11, B7/B44) were incubated for 5 hours in the tube thus obtained.

After this incubation, the whole blood was centrifuged for 10 min at 1500 rpm and the supernatant (plasma) from the blood was removed. The plasma (100, 50 and 12.50 microliters) was deposited in duplicate onto a 96-well plate pre-incubated with an anti-gamma-IFN antibody, for 2 h at a temperature of 20° C. After washing, a biotinylated anti-gamma-IFN 2^{nd} antibody was added for 1 h at a temperature of 20° C. Extravidin-AP was then added for 1 h at a temperature of 20° C., and 100 microliters of MUP substrate (substrate for alkaline phosphatase) made it possible to develop the reaction. Reading was carried out after 30 min.

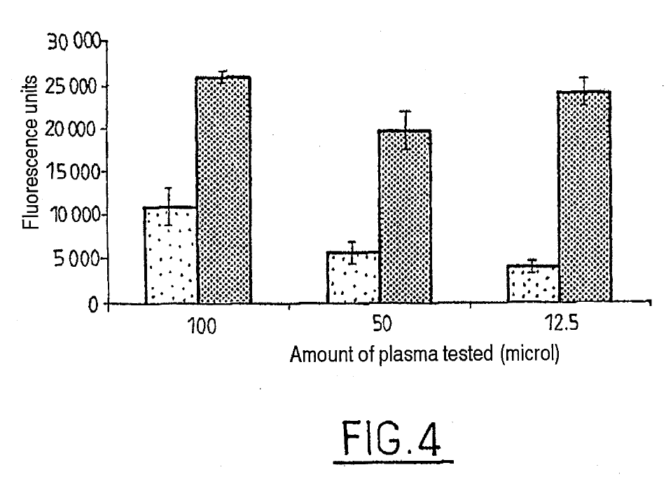

The results are given in FIG. 4.

Legend for FIG. 4:

[symbol] : −without peptide

[symbol] : 10 micrograms of peptide

These results show that it is possible to detect the presence of a cellular response specific for a virus (here detection of the EBV virus) using the whole blood of a patient. This cellular response is specific for the virus and the production of gamma-IFN was detected at various dilutions of plasma. The differential between the production of gamma-IFN obtained from the whole blood alone (negative control) or from the whole blood incubated with the EBV virus peptides is significant. The cells present in the blood of patient 1056 were activated and produced gamma-IFN in a specific manner in the presence of the EBV virus-derived CD8⁺ peptides.

1. The use of a solid support to which at least one peptide constituting a T epitope of an antigen is attached, for detecting and/or characterizing, in vitro, the cellular immunity with respect to said antigen.

2. A method for detecting and/or characterizing, in vitro, the cellular immune response of an individual with respect to an antigen, by bringing a biological sample comprising CD4⁺ T lymphocytes and/or CD8⁺ T lymphocytes present in the peripheral blood mononuclear cells of said individual into contact with one or more peptide(s) constituting a T epitope or T epitopes of said antigen capable of being presented by an MHC molecule of said individual, and detecting the effector T lymphocytes activated by this bringing into contact, which method is characterized in that said peptide(s) is(are) attached to a solid support.

3. The method as claimed in claim 2, wherein at least two peptides constituting two different T epitopes of said antigen, and at least one of which is capable of being presented by an MHC molecule of said individual, are attached to said solid support.

4. The method as claimed in claim 3, wherein a mixture of said peptides is attached to said solid support.

5. The method as claimed in claim 3, wherein said solid support consists of a microtitration plate, said peptides being attached in at least two different wells of said plate, and the biological sample is divided up into at least two aliquots, each of which is brought into contact with one of said peptides.

6. The method as claimed in claim 2, wherein the peptide(s) used constitute(s) a CD4⁺ T epitope or CD4⁺ T epitopes.

7. The method as claimed in claim 2, wherein the peptide(s) used constitute(s) a CD8⁺ T epitope or CD8⁺ T epitopes.

8. A kit for carrying out a method as claimed in claim 2, comprising:
an assortment of peptides derived from the same antigen, each of which constitutes an identified T epitope of said antigen, attached to a solid support, and;
means for detecting the activated effector T lymphocytes.

9. A kit for carrying out a method as claimed claim 2, comprising an assortment of peptides derived from the same antigen, each of which constitutes an identified T epitope of the said antigen, attached to a solid support, said assortment comprising at least one peptide constituting a CD4⁺ T epitope and at least one peptide constituting a CD8⁺ T epitope.
